(19) United States
(12) Patent Application Publication
(10) Pub. No.: US 2004/0126865 A1
(43) Pub. Date: Jul. 1, 2004

(54) Nucleic acids encoding ranpirnase variants and methods of making them

(75) Inventor: Shailendra K. Saxena, West Orange, NJ (US)

Correspondence Address: MARK H JAY, POST OFFICE BOX E, SHORT HILLS, NJ, 07078

(73) Assignee: ALFACELL CORPORATION

(21) Appl. No.: 10/331,910

(22) Filed: Dec. 30, 2002

(51) Int. Cl. C12N 9/22; C07H 21/04; C12N 1/21; C12N 15/74

(52) U.S. Cl. 435/199; 435/69.1; 435/252.33; 435/320.1; 536/23.2

(57) ABSTRACT

Ribonuclease DNA coding for an amino acid sequence beginning with a residue of glutamine is introduced into a vector of pET22b(+) plasmid to form recombinant plasmid DNA that begins with a Pel B leader sequence. The recombinant plasmid DNA is used to transform an E.coli BL21(DE3) host. Signal peptidase enzyme present in the host cell cleaves the pelB leader sequence during signal processing and thereby allows the glutamine residue to autocyclize to pyroglutamic acid. In this way, the Ribonuclease can be produced directly, i.e. without a separate step of cleaving the initial N-terminal methionine residue.

### BACKGROUND OF THE INVENTION

The invention relates to Ribonucleases (RNases), and more particularly relates to ranpirnase. In its most immediate sense, the invention relates to nucleic acids that encode proteins that produce ranpirnase and proteins closely related to ranpirnase (such closely related proteins being herein referred to as “variants” and “ranpirnase variants”).

Ranpirnase is the generic name of an RNase that is produced by Alfacell Corporation (assignee herein) under the registered trademark ONCONASE. Ranpirnase is a protein 104 residues long, with a blocked N-terminal of pyroglutamic acid (<Glu) that is produced by autocyclization of glutamine (Gln). Ranpirnase is disclosed in U.S. Pat. No. 5,519,212. Ranpirnase has demonstrated activity against certain lines of cancer cells, and is now being tested in human clinical trials.

Ranpirnase variants are also of clinical interest. U.S. Pat. No. 6,239,257 B1 discloses variants that are active against certain lines of cancer cells. And, U.S. Pat. No. 6,175,003 B1 discloses a cysteinized variant designed for specific conjugation to a targeting moiety.

U.S. Pat. No. 6,175,003 B1 discloses two nucleic acids that, after expression and purification followed by in vitro cleavage of an N-terminal residue of methionine at position −1, produce recombinant ranpirnase and a recombinant cysteinized ranpirnase, respectively. This cleavage allows a glutamine residue to autocyclize into pyroglutamic acid, which has been shown to be necessary for the activity of ranpirnase and is believed necessary for the activity of ranpirnase variants.

It would be advantageous to produce ranpirnase and ranpirnase variants directly, i.e. without the need for a separate in vitro step to cleave an N-terminal methionine residue.

Consequently, one object of the invention is to provide materials and methods that can be used to produce ranpirnase and ranpirnase variants.

The invention proceeds from the realization that a pET22b(+) vector has a built-in pelB signal peptide sequence (a “leader sequence”) that is cleaved co-translationally during expression of the desired gene. This co-translational cleavage is carried out by a signal peptidase enzyme present in the host cell, thereby avoiding the need for a separate in vitro cleavage step. More specifically, a ranpirnase gene (or the gene of a ranpirnase variant) having a codon for an N-terminal glutamine as the first amino acid is fused to an upstream pelB leader sequence of a pET22b(+) vector. Then, the resulting plasmid is expressed in an expression host (which is advantageously E.coli BL21(DE3) competent cells). The expressed protein is initially synthesized with a pelB leader sequence at its N-terminal. But, this pelB leader sequence does not remain. This is because the host bacterial cell contains signal peptidase enzyme. As the protein is exported to periplasm, the signal peptidase enzyme cleaves the N-terminal pelB leader sequence. This in turn permits glutamine, which is the first amino acid of the expressed protein (ranpirnase or a ranpirnase variant) to autocyclize into pyroglutamic acid at the N-terminal. In this way, autocyclization of the glutamine residue to pyroglutamic acid takes place spontaneously, and no separate cleavage step is required.

Hence, in accordance with the invention, plasmids are disclosed that, when expressed in an expression host, encode Ribonucleases in which glutamine is the N-terminal residue. Then, the N-terminal glutamine autocyclizes to form pyroglutamic acid. Advantageously, the expression host is E.coli BL21(DE3) competent cells, and the genes (of ranpirnase or ranpirnase variants) are cloned at the MscI and BamHI restriction enzyme sites in a pET22b(+) plasmid vector.

### BRIEF DESCRIPTION OF THE DRAWINGS

The invention will be better understood with the aid of the following illustrative and non-limiting drawings, in which:

FIG. 1 is a flowchart showing a first preferred embodiment of the invention;

FIG. 2 is a flowchart showing a second preferred embodiment of the invention; and

FIG. 3 is a flowchart showing a third preferred embodiment of the invention.

### DETAILED DESCRIPTION OF PREFERRED EMBODIMENTS

U.S. Pat. No. 6,175,003 B1 discloses two recombinant plasmids that are used as starting materials in accordance with the preferred embodiments of this invention. The first plasmid is named pET11d-rOnc(Q1, M23L, S72C), which is made up of the rOnc(Q1, M23L, S72C) gene cloned in a pET-11d vector. This first plasmid encodes a protein having an N-terminal methionine residue at position −1 and a residue of glutamine at position 1. From position 2 on, this first plasmid encodes a protein having the same amino acid sequence as ranpirnase, except that the methionine (Met) residue located at position 23 in ranpirnase is changed to a residue of leucine (Leu) and the serine (Ser) residue at position 72 of ranpirnase is changed to a residue of cysteine (Cys). The amino acid sequence of this encoded protein is shown in SEQ ID NO:1, and the nucleotide sequence for this encoded protein is SEQ ID NO:4. Reagents from Perkin Elmer (Branchburg N.J.), Stratagene (La Jolla Calif.) and Novagen (Madison Wis.) were used for PCR and other recombinant DNA manipulations.

In accordance with the first preferred embodiment, two primers are designed for use in a PCR protocol. These are a forward PCR primer (SEQ ID NO:2) and a reverse PCR primer (SEQ ID NO:3). The forward PCR primer generates a blunt 5′ end and the reverse PCR primer contains a stop codon followed by a BamHI restriction site at the 3′ end.

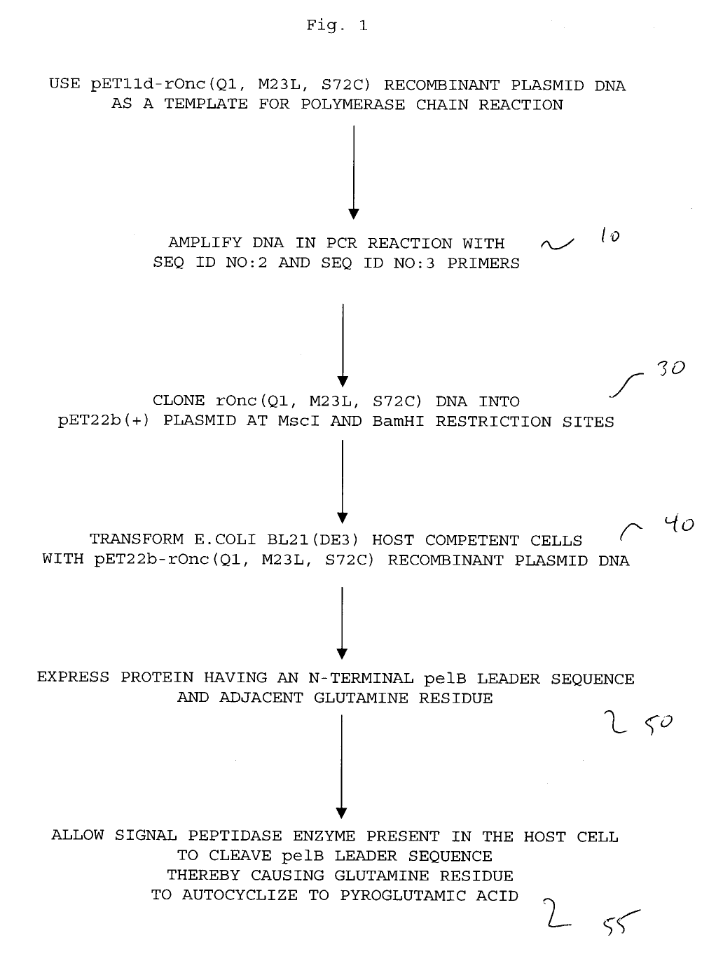

These primers are used to amplify a template of recombinant plasmid pET11d-rOnc(Q1, M23L, S72C) DNA (FIG. 1, step 10) in a PCR amplification reaction using Pfu DNA polymerase. The PCR reaction produces a full-length gene having a blunt 5′ end and a BamHI restriction site at the 3′ end. This new gene has been named rOnc(Q1, M23L, S72C). This gene can then be digested with BamHI restriction enzyme and cloned (FIG. 1, step 30) into a pET22b(+) plasmid vector at the MscI and BamHI restriction sites. The resulting pET22b-rOnc(Q1, M23L, S72C) recombinant plasmid DNA is then used to transform an expression host (FIG. 1, step 40). An appropriate host is E.coli BL21(DE3) competent cells. A protein is expressed from the host (FIG. 1, step 50). The expressed protein has an N-terminal pelB leader sequence followed by a residue of glutamine (Gln) at position 1. A signal peptidase enzyme present in the host cell cleaves the pelB leader sequence during signal processing, which allows the glutamine residue to autocyclize into pyroglutamic acid (<Glu) (FIG. 5, step 55).

The second preferred embodiment of the invention uses a second recombinant plasmid disclosed in the above-referenced '003 patent, namely pET11d-rOnc(Q1). This recombinant plasmid DNA is subjected to site-directed mutagenesis. This is because the pET11d-rOnc(Q1) recombinant plasmid DNA encodes a protein having a serine (Ser) residue at position 72 in its amino acid sequence, and in the second preferred embodiment the amino acid sequence of the encoded RNase is cysteinized by changing this serine residue to cysteine (Cys). (The amino acid sequence encoded by this second preferred embodiment is SEQ ID NO:5, and the nucleotide sequence for the encoded protein is SEQ ID NO:6.)

The primers used in the second preferred embodiment are chosen to achieve two objectives. As in the first preferred embodiment, the second preferred embodiment is designed to clone a gene into pET22b(+) plasmid vector at its MscI and BamHI restriction sites, and this objective is achieved by using the same forward and reverse primers (SEQ ID NO:2 and SEQ ID NO:3 respectively).

Additionally, the above-described site-directed mutagenesis is carried out using a mutated forward PCR primer SEQ ID NO:7 and a mutated reverse PCR primer SEQ ID NO:8. As in the first preferred embodiment, the forward PCR primer produces a blunt 5′ end, the reverse PCR primer contains a stop codon followed by a BamHI restriction site at the 3′ end (to permit cloning in the pET22b(+) plasmid vector at its MscI and BamHI restriction sites). The mutated forward and reverse PCR primers carry out the mutation of position 72 from serine (Ser) to cysteine (Cys).

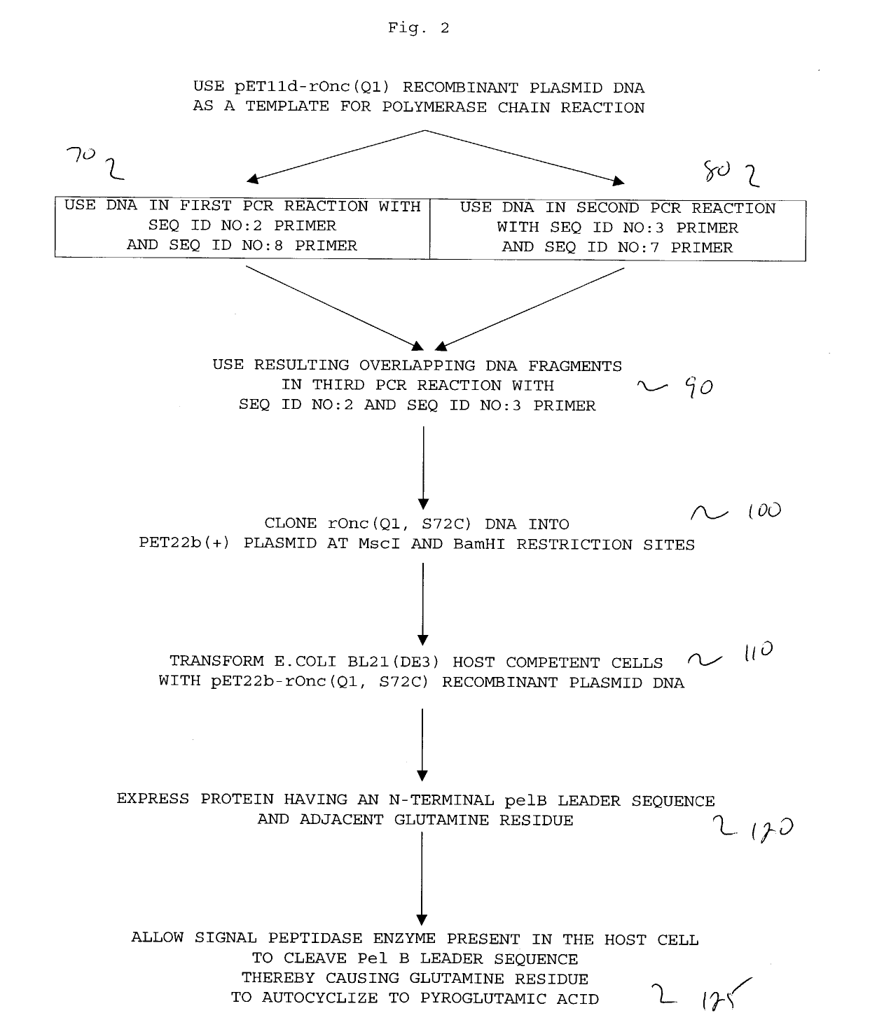

In a first PCR reaction using Pfu DNA polymerase (FIG. 2, step 70) the recombinant plasmid pET11d-rOnc(Q1) DNA is used as a template with the forward PCR primer SEQ ID NO: 2 and the mutated reverse PCR primer SEQ ID NO:8. In a second PCR reaction using Pfu DNA polymerase (FIG. 2, step 80), the recombinant plasmid pET11d-rOnc(Q1) DNA is used as a template with the reverse PCR primer SEQ ID NO:3 and the mutated forward PCR primer SEQ ID NO:7. These first and second PCR reactions produce overlapping DNA fragments that have the desired mutation (serine residue to cysteine residue at location 72). Then, in a third PCR reaction using Pfu DNA polymerase (FIG. 2, step 90), these overlapping DNA fragments are mixed together with the forward PCR primer SEQ ID NO:2 and the reverse PCR primer SEQ ID NO:3. This produces a full-length gene having a blunt 5′ end and a stop codon flanked by a BamHI restriction site at the 3′ end. This full-length gene has been named rOnc(Q1, S72C). The new rOnc(Q1, S72C) gene can then be digested with BamHI restriction enzymes and cloned (FIG. 2, step 100) in pET22b(+) plasmid at the MscI and BamHI restriction sites to produce a pET22b-rOnc(Q1, S72C) recombinant plasmid. The resulting pET22b-rOnc(Q1, S72C) recombinant plasmid is then used to transform E.coli BL21(DE3) competent cells (FIG. 2, step 110). A protein is expressed from the host (FIG. 2, step 120). The expressed protein has an N-terminal pelB leader sequence followed by a residue of glutamine (Gln) at position 1. A signal peptidase enzyme present in the host cell cleaves the pelB leader sequence during signal processing, which allows the glutamine residue to autocyclize into pyroglutamic acid (<Glu) (FIG. 2, step 125).

The third preferred embodiment of the invention uses the above-referenced second recombinant plasmid, namely pET11d-rOnc(Q1), to produce an amino acid sequence that encodes ranpirnase, which is SEQ ID NO:9. The nucleotide sequence for ranpirnase is SEQ ID NO:10.

The third preferred embodiment is also designed to be cloned into pET22b(+) plasmid vector at its MscI and RamHI restriction sites, and it therefore uses the same forward PCR primer (SEQ ID NO:2) and reverse PCR primer (SEQ ID NO:3) as were used in the first and second preferred embodiments.

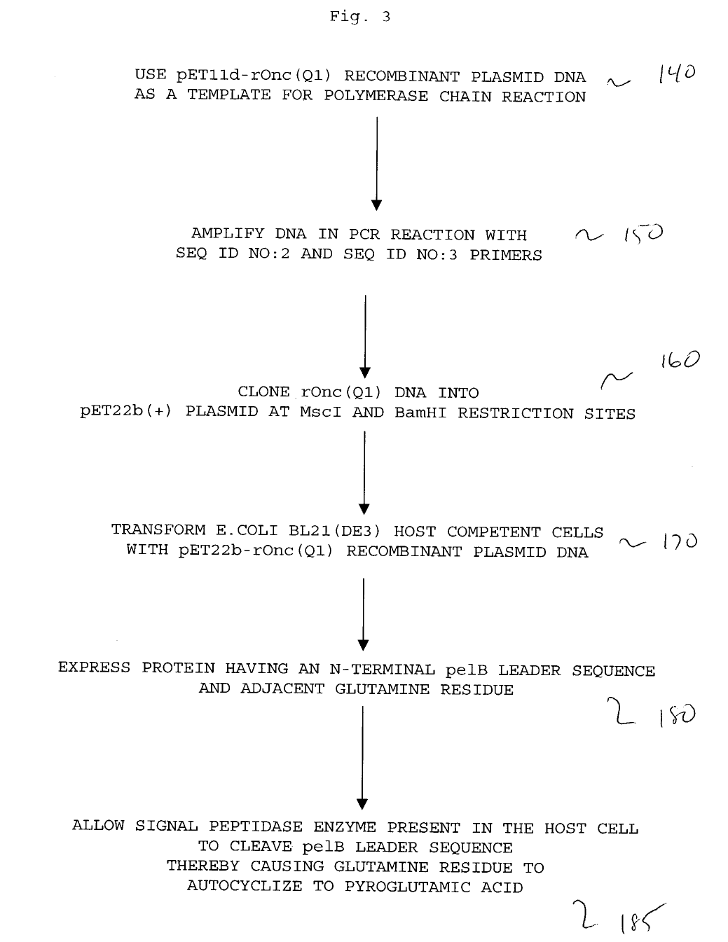

These primers are used in a PCR amplification reaction using Pfu DNA polymerase. The recombinant plasmid pET11d-rOnc(Q1) DNA (FIG. 3, step 140) is used as a template with the forward PCR primer SEQ ID NO:2 and the reverse PCR primer SEQ ID NO:3 (FIG. 3, step 150). This produces a full-length gene having a blunt 5′ end and a BamHI restriction site at the 3′ end. This new gene has been named rOnc(Q1). This gene can then be digested with BamHI restriction enzyme and cloned (FIG. 3, step 160) into a pET22b(+) plasmid vector at the MscI and BamHI restriction sites. The resulting pET22b-rOnc(Q1) recombinant plasmid DNA is then used to transform an expression host (FIG. 3, step 170). An appropriate host is E.coli BL21(DE3) competent cells. A protein is expressed from the host (FIG. 3, step 180). The expressed recombinant protein has an N-terminal pelB leader sequence followed by glutamine (Gln). A signal peptidase enzyme present in the host cleaves the pelB leader sequence during signal processing, which allows the glutamine (Gln) residue to autocyclize to form pyroglutamic acid (<Glu) (FIG. 3, step 185).

Persons skilled in the art will recognize that as a practical matter it is impossible to insure that all the initial glutamine residues actually autocyclize to pyroglutamic acid. For this reason, in each of these preferred embodiments, the end product of the identified steps is actually a mixture of proteins. In the first preferred embodiment, the end product is actually a mixture of the protein of SEQ ID NO:1 and a cyclized form of the protein of SEQ ID NO:1 in which the N-terminal residue is pyroglutamic acid. Similarly, in the second preferred embodiment, the end product is actually a mixture of the protein of SEQ ID NO:5 and a cyclized form of the protein of SEQ ID NO:5 in which the N-terminal residue is pyroglutamic acid. So, too, in the third preferred embodiment, the end product is actually a mixture of the protein of SEQ ID NO:9 and a cyclized form of the protein of SEQ ID NO:9 in which the N-terminal residue is pyroglutamic acid. The proteins in which the initial residue remains as glutamine can either be removed by purification or, alternatively, the initial glutamine residues can be converted to pyroglutamic acid.

In these preferred embodiments, the PCR reactions are carried out using Pfu DNA polymerase, the expression host is E.coli BL21(DE3) competent cells, the vector is pET22b(+) plasmid, and the new genes are cloned into the vector at the MscI and BamHI restriction sites. While the use of Pfu DNA polymerase, E.coli BL21(DE3), pET22b(+) plasmid, and the MscI and BamHI restriction sites are all preferred, they are not necessary to the invention. Other polymerases, hosts, plasmids, and restriction sites can be used instead.

Likewise, the primers used in all the herein-disclosed embodiments generate full length DNA having a blunt 5′ end and a stop codon flanked by a BamHI site at the 3′ end. This is because all the herein-disclosed embodiments are designed to permit DNA to be cloned into pET22b(+) plasmid vector at its MscI and BamHI sites. Although such primers are preferred, they are not necessary to the invention. If another vector were to be used, or if other sites in pET22b(+) plasmid were to be used, the primers would be changed appropriately.

Persons skilled in the art will recognize that for clarity, certain details known to persons skilled in the art have been omitted from this description. For example, after a gene has been cloned into the preferred pET22b(+) vector as disclosed herein and E.coli BL21(DE3) competent host cells have been transformed with recombinant DNA therein, the host cells must be induced with IPTG in order to express a Ribonuclease, as desired. Of course, if other vector and/or host cells were to be used, other materials would be used to induce them.

Persons skilled in the art will recognize that conservative modifications of the herein-disclosed genes and proteins are within the scope of the invention. Insofar as conservative modification of genes is concerned, this is because the genetic code is degenerate and different codons encode the same amino acid residue. Insofar as conservative modification of proteins is concerned, this is because different amino acid residues can have very similar characteristics and conservatively modified proteins can have the same or highly similar properties.

Although at least one preferred embodiment of the invention has been described above, this description is not limiting and is only exemplary. The scope of the invention is defined only by the following claims:

1. A plasmid containing the gene of SEQ ID NO:4 that, when expressed in a host, encodes a precursor form of the Ribonuclease of SEQ ID NO:1, said precursor form having an N-terminal glutamine residue that autocyclizes to pyroglutamic acid.

2. The plasmid of claim 1, wherein the expression host is E.Coli BL21(DE3).

3. A plasmid containing the gene of SEQ ID NO:6 that, when expressed in a host, encodes a precursor form of the Ribonuclease of SEQ ID NO:5, said precursor form having an N-terminal glutamine residue that autocyclizes to pyroglutamic acid.

4. The plasmid of claim 3, wherein the expression host is E.coli BL21(DE3).

5. A plasmid containing the gene of SEQ ID NO:10 that, when expressed in a host, encodes a precursor form of the Ribonuclease of SEQ ID NO:9, said precursor form having an N-terminal glutamine residue that autocyclizes to pyroglutamic acid.

6. The plasmid of claim 5, wherein the expression host is E.coli BL21(DE3).

7. A recombinantly produced mixture of proteins obtained by expressing the gene of SEQ ID NO:4 in an E.coli BL21(DE3) host, wherein one of the proteins in the mixture is the protein of SEQ ID NO:1 and another one of the proteins in the mixture is a cyclized form of the protein of SEQ ID NO:1, said cyclized form having an N-terminal residue of pyroglutamic acid.

8. A recombinantly produced mixture of proteins obtained by expressing the gene of SEQ ID NO:6 in an E.coli BL21(DE3) host, wherein one of the proteins in the mixture is the protein of SEQ ID NO:5 and another one of the proteins in the mixture is a cyclized form of the protein of SEQ ID NO:5, said cyclized form having an N-terminal residue of pyroglutamic acid.

9. A recombinantly produced mixture of proteins obtained by expressing the gene of SEQ ID NO:10 in an E.coli BL21(DE3) host, wherein one of the proteins in the mixture is the protein of SEQ ID NO:9 and another one of the proteins in the mixture is a cyclized form of the protein of SEQ ID NO:9, said cyclized form having an N-terminal residue of pyroglutamic acid.

10. A plasmid that, when expressed in an E.coli host, encodes a conservatively modified variant of the Ribonuclease of SEQ ID NO:1, said conservatively modified variant having an N-terminal residue of glutamine that autocyclizes to pyroglutamic acid.

11. A plasmid that, when expressed in an E.coli host, encodes a conservatively modified variant of the Ribonuclease of SEQ ID NO:5, said conservatively modified variant having an N-terminal residue of glutamine that autocyclizes to pyroglutamic acid.

12. A plasmid that, when expressed in an E.coli host, directly encodes a conservatively modified variant of the Ribonuclease of SEQ ID NO:9, said conservatively modified variant having an N-terminal residue of glutamine that autocyclizes to pyroglutamic acid.

13. A method of recombinantly producing the Ribonuclease of SEQ ID NO:1, comprising the following steps:
starting with a plasmid vector having an N-terminal pelB leader sequence followed by the gene of SEQ ID NO:4;
expressing the gene in a host, thereby producing a Ribonuclease; and
allowing the pelB leader sequence to be co-translationally cleaved during signal processing by signal peptidase enzyme that is present in the host,
whereby an initial N-terminal residue of glutamine in said Ribonuclease autocyclizes to pyroglutamic acid.

14. A method of recombinantly producing the Ribonuclease of SEQ ID NO:5, comprising the following steps:
starting with a plasmid vector having an N-terminal pelB leader sequence followed by the gene of SEQ ID NO:6;
expressing the gene in a host, thereby producing a Ribonuclease; and
allowing the pelB leader sequence to be co-translationally cleaved during signal processing by signal peptidase enzyme that is present in the host, whereby an initial N-terminal residue of glutamine in said Ribonuclease autocyclizes to pyroglutamic acid.

15. A method of recombinantly producing the Ribonuclease of SEQ ID NO:9, comprising the following steps:
starting with a plasmid vector having an N-terminal pelB leader sequence followed by the gene of SEQ ID NO:10;
expressing the gene in a host, thereby producing a Ribonuclease; and
allowing the pelB leader sequence to be co-translationally cleaved during signal processing by signal peptidase enzyme that is present in the host, whereby an initial N-terminal residue of glutamine in said Ribonuclease autocyclizes to pyroglutamic acid.

16. A method of constructing the gene of SEQ ID NO:4, which gene encodes the Ribonuclease of SEQ ID NO:1, comprising the following steps:
using pET11d-rOnc (Q1, M23L, S72C) recombinant plasmid DNA as a template for amplification in a polymerase chain reaction; and
cloning the DNA into a pET-22b(+) plasmid vector.

17. The method of claim 16, wherein said cloning step is carried out by digesting the DNA with BamHI restriction enzyme and introducing it at the MscI and BamHI restriction sites of the pET-22b(+) plasmid.

18. A method of constructing the gene of SEQ ID NO:6, which gene encodes the Ribonuclease of SEQ ID NO:5, comprising the following steps:
starting with pET11d-rOnc (Q1) recombinant plasmid DNA as a template;
using site-directed mutagenesis to substitute a cysteine residue for the serine residue at position 72 of the DNA, thereby producing full-length mutated DNA; and
cloning the mutated DNA into a pET-22b(+) plasmid vector.

19. The method of claim 18, wherein said cloning step is carried out by digesting the mutated DNA with BamHI restriction enzyme and introducing it at the MscI and BamHI restriction sites of the pET-22b(+) plasmid.

20. A method of constructing the gene of SEQ ID NO:10, which gene encodes the Ribonuclease of SEQ ID NO:9, comprising the following steps:
using pET11d-rOnc (Q1) recombinant plasmid DNA as a template for amplification in a polymerase chain reaction; and
cloning the DNA into a pET-22b(+) plasmid vector.

21. The method of claim 20, wherein said cloning step is carried out by digesting the DNA with BamHI restriction enzyme and introducing it at the MscI and BamHI restriction sites of the pET-22b(+) plasmid.

22. A vector that, when expressed in an E.coli host, encodes a Ribonuclease having an N-terminal residue of glutamine that autocyclizes to form pyroglutamic acid.

23. The vector of claim 22, wherein said Ribonuclease is the Ribonuclease of SEQ ID NO:1.

24. The vector of claim 22, wherein said Ribonuclease is the Ribonuclease of SEQ ID NO:5.

25. The vector of claim 22, wherein said Ribonuclease is the Ribonuclease of SEQ ID NO:9.


SEQUENCE LISTING (10 sequences)

SEQ ID NO: 1 - protein, 104 aa, Artificial Sequence
  Protein encoded by pET22B-rOnc(Q1, M23L, S72C) DNA
QDWLTFQKKHITNTRDVDCDNILSTNLFHCKDKNTFIYSRPEPVKAICKGIIASKNVLTTSEFYLSDCNVTCRPCKYKLKKSTNKFCVTCENQAPVHFVGVGSC

SEQ ID NO: 2 - DNA, 29 bp, Artificial Sequence
  Forward PCR primer
cccaggactggctgactttccagaaaaaa

SEQ ID NO: 3 - DNA, 32 bp, Artificial Sequence
  Reverse PCR primer
cgcgcggatccctactagcaagaaccaacacc

SEQ ID NO: 4 - DNA, 312 bp, Artificial Sequence
  Nucleotide sequence of rOnc(Q1, M23L, S72C) DNA
caggactggctgactttccagaaaaaacatatcactaacactcgtgacgttgactgcgacaacatcctgtctactaacctgttccattgcaaagacaaaaacactttcatctactctcgtccggaaccggttaaagctatctgcaaaggtatcatcgcttctaaaaacgttctgactacttctgaattctacctgtctgactgcaacgttacttgccgtccgtgcaaatacaaactgaaaaaatctactaacaaattctgcgttacttgcgaaaaccaggctccggttcatttcgttggtgttggttcttgc

SEQ ID NO: 5 - protein, 104 aa, Artificial Sequence
  Protein encoded by pET22b-rOnc(Q1, S72C) DNA
QDWLTFQKKHITNTRDVDCDNIMSTNLFHCKDKNTFIYSRPEPVKAICKGIIASKNVLTTSEFYLSDCNVTCRPCKYKLKKSTNKFCVTCENQAPVHFVGVGSC

SEQ ID NO: 6 - DNA, 312 bp, Artificial Sequence
  Nucleotide sequence of rOnc(Q1, S72C) DNA
caggactggctgactttccagaaaaaacatatcactaacactcgtgacgttgactgcgacaacatcatgtctactaacctgttccattgcaaagacaaaaacactttcatctactctcgtccggaaccggttaaagctatctgcaaaggtatcatcgcttctaaaaacgttctgactacttctgaattctacctgtctgactgcaacgttacttgccgtccgtgcaaatacaaactgaaaaaatctactaacaaattctgcgttacttgcgaaaaccaggctccggttcatttcgttggtgttggttcttgc

SEQ ID NO: 7 - DNA, 39 bp, Artificial Sequence
  Mutated forward PCR primer
gactgcaacgttacttgccgtccgtgcaaatacaaactg

SEQ ID NO: 8 - DNA, 39 bp, Artificial Sequence
  Mutated reverse PCR primer
gtatttgcacggacggcaagtaacgttgcagtcagacag

SEQ ID NO: 9 - protein, 104 aa, Artificial Sequence
  Recombinant ranpirnase
QDWLTFQKKHITNTRDVDCDNIMSTNLFHCKDKNTFIYSRPEPVKAICKGIIASKNVLTTSEFYLSDCNVTSRPCKYKLKKSTNKFCVTCENQAPVHFVGVGSC

SEQ ID NO: 10 - DNA, 312 bp, Artificial Sequence
  Ranpirnase gene
caggactggctgactttccagaaaaaacatatcactaacactcgtgacgttgactgcgacaacatcatgtctactaacctgttccattgcaaagacaaaaacactttcatctactctcgtccggaaccggttaaagctatctgcaaaggtatcatcgcttctaaaaacgttctgactacttctgaattctacctgtctgactgcaacgttacttctcgtccgtgcaaatacaaactgaaaaaatctactaacaaattctgcgttacttgcgaaaaccaggctccggttcatttcgttggtgttggttcttgc